(19) United States
(12) Patent Application Publication
(10) Pub. No.: US 2006/0107431 A1
(43) Pub. Date: May 25, 2006

(54) Supplied air helmet having a knitted face seal

(76) Inventors: Desmond T. Curran, County Durham (GB); Peter D. Lee, Cleveland (GB); Richard D. Williams, Lancaster (GB); Peter N. Yates, Lancaster (GB)

Correspondence Address: 3M INNOVATIVE PROPERTIES COMPANY, PO BOX 33427, ST. PAUL, MN, 55133-3427, US

(21) Appl. No.: 10/987,641

(22) Filed: Nov. 12, 2004

(51) Int. Cl. A42B 1/00; A62B 18/00; A61F 9/00; A62B 17/04

(52) U.S. Cl. 2/7; 128/201.24

(57) ABSTRACT

A supplied air helmet 10 that has a visor 14 and a face seal 16. The face seal 16 is secured to the visor 14 and includes a sealing member 18 that comprises a knitted fabric. The knitted fabric is disposed on the face seal 16 in a location where the face seal 16 makes contact with a wearer's face. The use of a knitted fabric for the face seal 16 makes the supplied air helmet 10 more comfortable to wear and makes it more likely that helmet wearers will consistently use the face seal 16 while donning the supplied air helmet 10.

The present invention pertains to the use of a knitted face seal on a supplied air helmet.

### BACKGROUND

Supplied air helmets are regularly worn in environments where the surrounding air contains contaminants. These helmets have a fluid impermeable visor that is located in front of the wearer's face when the helmet is worn. The visor has a window through which the wearer can see the surrounding environment. A face seal is attached to the visor to provide a breathing zone or an interior gas space that is separate from the surrounding exterior gas space. The interior gas space is located between the visor and the wearer's face and is defined, for the most part, by the face seal that is attached to the visor, the visor body, and the wearer's face. U.S. Pat. Nos. 6,014,971, 4,462,399, and 4,280,491 disclose examples of supplied air helmets that have face seals.

Clean air is forced into the interior gas space from a supply tank or from a powered air system that drives ambient air through an air filter. The wearer breathes this clean air and exhales it back into the breathing zone. This exhaled air, along with excess clean air that is forced into the breathing zone from the supply source, exits the interior gas space through openings in the face seal. The positive pressure that occurs within the interior gas space precludes contaminants from entering the interior gas space through the face seal openings. Welders, for example, often use supplied air helmets as protection from breathing contaminants generated during welding procedures. Examples of welding helmets are shown in the following patent documents: U.S. Pat. Nos. 6,557,174, 6,591,424, 6,185,739, 5,533,206, 5,191,468, 5,140,707, 4,875,235, 4,853,973, 4,774,723, 4,011,594 and Des. 398,421; and WO 00/59421 and WO 99/26502.

Known face seals have been made from a variety of materials. Some supplied air helmets have used Tyvek™ or Sontara™ from DuPont as the face seal material—see U.S. Pat. No. 6,250,299 B1 to Danisch et al. and U.S. Pat. No. 6,016,805 to Burns et al., respectively. Others have used a soft pad or foam material—see U.S. Pat. Nos. 5,533,500 and 5,104,430 to Her-Mou and U.S. Pat. No. 5,054,479 to Yelland et al., respectively. Some commercially available products have used a PVC coated fabric as the face seal. Some fabric products also have incorporated an elastic material around the edge of the face seal to enable it to conform to different shaped faces. Other commercially available products such as the Performa A-VL face shield (available from North Safety Products of Middelburg, Netherlands) have used a woven fabric for the face seal. The woven fabric has had a free edge that extends radially inward from the visor to contact the wearer's face to create a breathing zone separate from the ambient air space.

While known face seal products have provided a boundary that precludes the helmet wearer from breathing contaminants that are present in the surrounding environment, these known products have sometimes created discomfort when contacting a wearer's face. The known face seals may create an itching sensation, they may be rough on the person's face, or they may not breathe properly when worn for extended periods. For these reasons, some users have removed the face seal or have not replaced it when its service life has ended. Non-use of a face seal can be hazardous to the wearer because contaminants can more easily enter the breathing zone. The present invention is directed toward alleviating the discomfort problem to ultimately improve end-user safety.

### SUMMARY OF THE INVENTION

The present invention addresses a need for face seal that can comfortably fit on various sized faces while permitting the rapid air exhaustion from within the interior gas space. Air that exits the interior gas space can readily enter the exterior gas space to allow room for new clean air. In brief summary, the present invention provides a supplied air helmet that comprises a visor and a face seal where the face seal is secured to the visor and includes a sealing member that comprises a knitted fabric. The knitted fabric is porous in at least some regions and is disposed on the face seal in a location where the sealing member makes contact with the wearer's face. Preferably, the knitted fabric has a fold that defines a radially inward periphery of the sealing member. The knitted porous fabric also preferably includes an elastic yarn in the knit itself to enable the sealing member to snugly fit against various sized faces.

The inventive supplied air helmet is beneficial in that the use of a knitted fabric in a face seal provides the wearer with extraordinarily improved comfort and fit while also enabling a controlled exhaustion of air from the interior gas space. The improved comfort is particularly beneficial because it improves the chances that the wearer will consistently use a face seal on the supplied air helmet when working in a contaminated environment, and the improved fit is beneficial in that it provides substantially less opportunity for the wearer to inhale contaminants that may be present in the ambient environment or exterior gas space. Ultimately, the invention is advantageous in that it may preserve the wearer's long term health.

These and other advantages of the invention are more fully shown and described in the drawings and detailed description of this invention, where like reference numerals are used to represent similar parts. It is to be understood, however, that the drawings and description are for the purposes of illustration only and should not be read in a manner that would unduly limit the scope of this invention.

### GLOSSARY

The terms set forth below will have the meanings as defined:

“breathing zone” means an interior gas space or a portion of the interior gas space where oxygen is inhaled by a wearer of a supplied air helmet;

“clean air” means air that has been filtered or that otherwise has been made safe to breathe;

“elastic” means the ability of a strained material (e.g. yarn or sealing member) to substantially recover its original size and shape immediately after being stressed to at least about twice its original length;

“exterior gas space” means the ambient atmospheric gas space that surrounds the exterior of a supplied air helmet when worn on a person;

“face” means the area on the front of a person's head, defined mainly by the cheeks and temporal area (or side portions of the facial region), chin, forehead, and facial area located therebetween;

“face seal” means a structure that contacts a person's face to help separate an interior gas space of a supplied air helmet from an exterior gas space;

“filtered air” means air that has been passed through a filter material to reduce the amount of any contaminants that may have been present in the air before it was filtered;

“frame member” means a structural part(s) that plays a role in supporting a sealing member;

“frictionally” means through use of friction;

“frictional engagement” means that the engagement occurs as a result of friction between the two parts intending to be joined without need for additional fastening from elements such as pegs, clips, and/or hook and loop materials;

“helmet” means a device that is worn on a person's head for safety and/or protection purposes;

“interior gas space” means the space that exists between a visor, a face seal, and a person's face when a supplied air helmet is being worn;

“knitted” means that the fabric is formed predominantly from a series of interlocked or interlocking loops that do not generally intersect each other perpendicularly in an over-and-under fashion;

“porous” means having fluid permeability sufficient to enable air in an interior gas space of a supplied air helmet to be purged or exhausted from that space to enter an exterior gas space while under pressure from an air or oxygen supply source;

“sealing member” means a structure or combination of parts that conformably contacts a person's face to assist in defining a separation between an interior gas space and an exterior gas space;

“supplied air helmet” means a helmet that receives a supply of clean air for a wearer of the device to breathe;

“visor” means a structure that is located in front of a person's face when worn and that has a window to allow the person to see through it;

“welding helmet” means a helmet that has a darkened or darkening window; and

“yarn” means a continuous strand of textile fiber(s), filament(s), or other material in a form suitable for knitting.

### BRIEF DESCRIPTION OF THE DRAWINGS

In the drawings:

FIG. 1 is a rear perspective view of a face seal 12 in accordance with the present invention;

FIGS. 2a-2d show various knitted patterns that could be used in connection with the present invention;

FIG. 2e shows an example of a plain weave;

FIG. 3 is a left side view of the face seal 12 shown in FIG. 1;

FIG. 4 is a cross-sectional view of the face seal 12, taken along lines 2-2 of FIG. 1;

FIG. 5 is a rear perspective view of a supplied air helmet 10 in accordance with the present invention, illustrating the face seal 12 displaced from the visor 14;

FIG. 6 is a left side view of a supplied air helmet 10, having an air duct 74;

FIG. 7 is a plan view of a knitted fabric 80 that may be used to form a series of blanks 82, which blanks 82, in turn, may be used to form a sealing member 18 (FIG. 1) for a face seal 12 (FIG. 1);

FIG. 8 is a front view of a segment 82 created from the knitted fabric 80 shown in FIG. 6; and

FIG. 9 is a front view of a sealing member blank 92 having a configuration that may be used to form a sealing member 18 (FIG. 1) for a face seal 12 (FIG. 1).

### DETAILED DESCRIPTION OF PREFERRED EMBODIMENTS

In describing preferred embodiments of the invention, specific terminology is used for the sake of clarity. The invention, however, is not intended to be limited to the specific terms so selected, and it is to be understood that each term so selected includes all the technical equivalents that operate similarly.

In the practice of the present invention, a new supplied air helmet is provided, which helmet offers improved comfort to the wearer to reduce opportunities for non-use of the face seal. The inventive supplied air helmet comprises a visor and a face seal. The face seal is secured to the visor and includes a sealing member that comprises a knitted porous fabric. The knitted porous fabric is located on the face seal where the sealing member makes contact with a wearer's face. The inventors discovered that a knitted porous fabric, particularly a fabric that includes an elastic yarn and/or a folded edge, provides a face seal that feels soft and comfortable when in contact with a person's face and that can allow excess air to be purged from the interior gas space of the helmet without allowing significant contaminant inflow. The knitted porous fabric also can be adapted to snugly fit various sized faces.

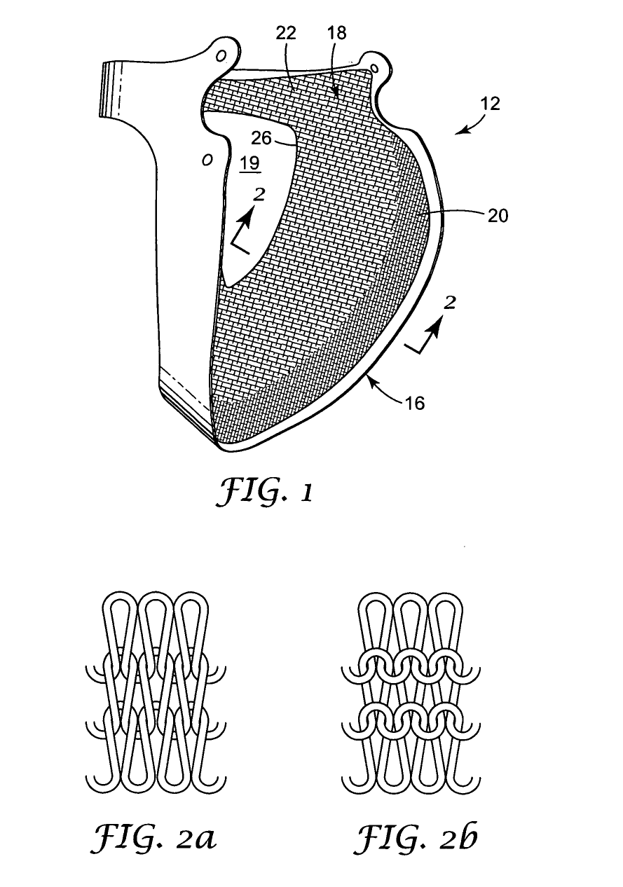

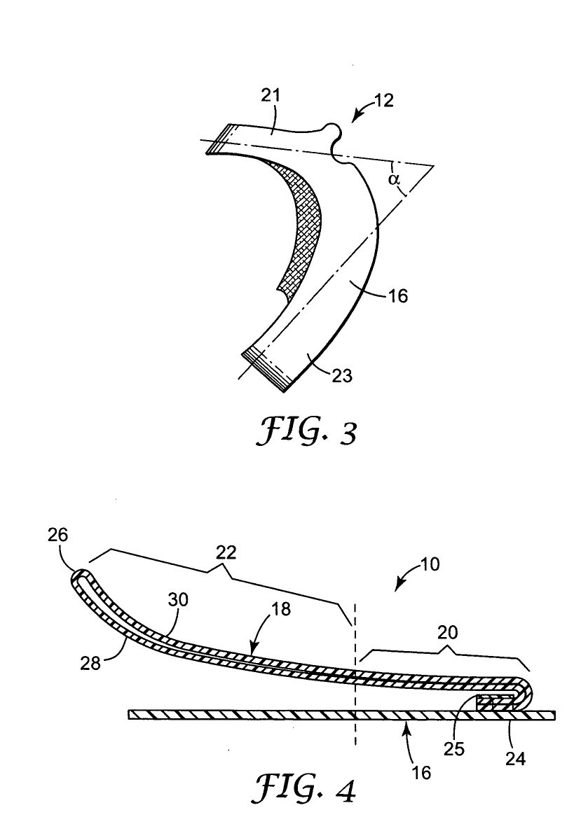

FIG. 1 illustrates a face seal 12 that includes a frame member 16 and a sealing member 18. The frame member 16 is made from a non-porous, or fluid impermeable, material such as a thin solid plastic. The frame member 16 provides structural integrity to, and support for, the face seal 12. Although the frame member 16 can be manually manipulated into position on the visor 14 (FIG. 4) in response to a force or pressure from a person's hands, it nonetheless generally returns to its original configuration when that manual pressure is removed. Thus, the frame member 16 is desirably flexible or manually conformable while still exhibiting sufficient structural properties to enable the face seal 12 to retain its desired sealing configuration when in use. In general, the plastic frame may be about 0.2 mm to 5 mm thick and can be structured such that it is not overly flimsy and such that it retains its intended shape when not being subjected to manual forces or pressure. The frame member 16 may be made of, for example, a 0.5 to 1.5 mm thick plastic such as polypropylene. The frame member 16 desirably has a matted surface on at least one side to avoid reflection that could interfere with a user's vision. Although the frame member is shown as a separate part, it could, alternatively, be integrally formed into the visor, and the sealing member could be secured to such an integral part. As the term is used in this document, “integral” means being formed as a single part rather than being created as a separate part that is subsequently joined together. An integral frame member may be constructed such that it is “one-and-the-same” with the visor body so as to be unnoticeable, or it could be a projecting flange or ridge that integrally extends from the visor body. Preferably, the frame member is formed as a separate part as shown in the drawings to enable the face seal to be more easily replaced.

FIG. 1 further shows that the sealing member 18 extends radially inward from the frame member 16 and has an opening 19 to accommodate a person's face. The opening 19 may be defined by the peripheral edge 26, which edge 26 is preferably elastically stretchable and is preferably sized to be smaller than the typical wearer's face to enable the face seal to fit snugly against the various sized faces. The knitted fabric preferably possesses elastic properties so that it can be stretched to tightly or snugly fit against a person's face. The stretchable properties allow the knitted fabric to make adequate contact with a person's face when the supplied air helmet is worn by the user and the visor 14 (FIG. 4) is pulled down in front of the wearer's face. These properties also enable the face seal to return to its original configuration when the helmet is not being worn. The elasticity of the face seal is preferably provided by including an elastic yarn in the face seal knit rather than sewing or otherwise securing a strip of elastic material to the face seal at, or in the vicinity of, the inner peripheral edge 26.

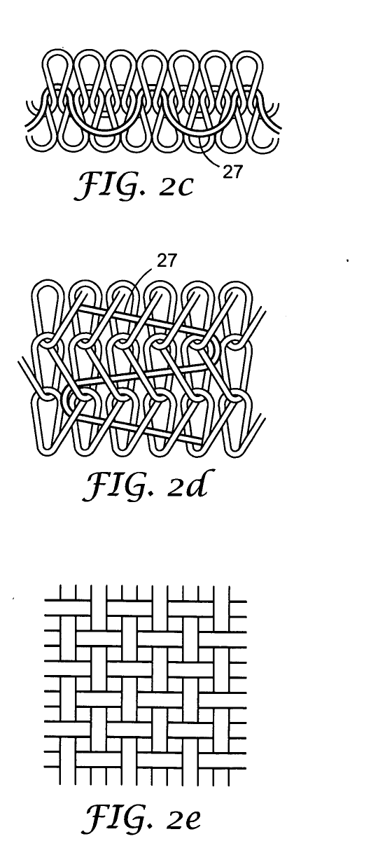

The knitted fabric is porous in at least some regions to allow pressurized fluid within the interior gas space to pass therethrough relatively unrestrained so that air that enters the breathing zone of the supplied air helmet can be exhausted into the environment to make way for new clean air that is forced into the breathing zone from an air supply source. In a supplied air system, the air pressure within the interior gas space is generally greater than the air pressure in the exterior gas space. This increased pressure causes air to be forced out from the interior gas space. The continuous forced influx of higher pressure clean air into the breathing zone creates an increased or pressurized environment within the interior gas space. The air flow can exit the interior gas space through the pores that may be present in portions of the knitted fabric. As FIGS. 2a-2d show, knitted fabrics are generally inherently (or integrally) porous, and this porosity allows air to escape from the interior gas space through the open-work porous knit. The knit may be achieved by knitting one or more yarns in such a fashion that a series of interlocked or interlocking loops are created within the knit. FIG. 2a shows a front face of a circular knit fabric, and FIG. 2b shows a rear view of that fabric. As shown, the knit includes a series of interlocking loops. A knitted fabric is distinguished from a purely woven or plain weave fabric (see FIG. 2e) where the yarn(s) go over-and-under yarns that run ninety degrees or generally perpendicular to them. Knitted fabrics thus comprise or consist essentially of a series of interlocking loops of one or more yarns where the yarns do not necessarily intersect each other perpendicularly in an over-and-under fashion. This construction tends to make knitted fabrics inherently more flexible than purely woven fabrics, a feature that may improve comfort and fit when used as a face seal. Knitted fabrics also generally can be stretched in both major dimensions of the predominantly two-dimensional structure. Knitted fabrics thus tend to be more malleable or conformable, which makes the face seal fit more snugly and comfortably to a wearer's face. FIGS. 2c and 2d show that additional yarns may be included in knitted fabrics. For example, in FIG. 2c, a laid-in yarn 27 may be included in the circular knit fabric. As shown in FIG. 2d, the laid-in yarn 27 can be included with a warp-knit fabric.

The knitted portion of the sealing member may comprise, for example, a combination of elastic yarn, flame-retardant yarn, and comfort yarns. The “elastic yarn” is used to improve the fabric's “stretchability”, the “flame-retardant yarn” functions to make the sealing member resist burning or deterioration from excess heat, and the “comfort yarn” improves the “feel” of the sealing member against a person's face. As indicated, the improved stretchability feature allows the sealing member to achieve a snug fit against various sized faces. The elastic yarn may be made predominantly from a polymer such as polyurethane or may be, for example, a modified acrylic, a latex, or a combination thereof. Commercially available products include Lycra™ (available from the DuPont Corporation of Wilmington, Del.), and Spandex™ (available from Invista Inc, of Wichita, Kans.). The flame-retardant feature is important in applications such as welding and grinding where the face seal can come into contact with sparks or drops of hot molten metal. The flame-retardant yarn may be made from a material that is inherently flame-retardant, or it may be treated, for example, chemically, to impart flame retardance to the fabric. An inherently flame-retardant material is generally preferred because it may have better wash durability. Examples of flame-retardant yarns may include oxidized thermally stabilized polycarylonitriles, flame-retardant polyester modified acrylics, and some nylons. Commercially available products include Kanecaron™ Protex-M (an inherently flame-retardant fiber available from the Kaneka Corporation, Osaka, Japan), Panox™ (available from Lantor Universal Carbon Fibres of Cleckheaton, UK), Nomex™ (available from the Du Pont Corporation), and Trevira™ (available from Trevira GmbH of Bobingen, Germany). The comfort yarn assists in furnishing the fabric with a feel that is soft to touch, thus making the fabric comfortable when in contact with a person's face. An example of a comfort yarn that may be used in the inventive face seal is cotton. Other suitable comfort yarns may include polyester, acrylic, rayon, and wool. The elastic, flame-retardant, and comfort yarns may generally be used in the fabric at about 0 to 20%, 30 to 100%, and to 70%, respectively, based on the weight of the fabric. Preferably, the elastic, flame-retardant, and comfort yarns are used at about 1 to 10%, 35 to 70%, and 30 to 60%, respectively. If the flame-retardant and comfort yarns are combined, they are used at about 80 to 99% by weight.

The fabric further can be essentially any color and can be made from dyed polymeric materials such as polyester, modified acrylic, or a mixture or blend of these polymeric materials with dyed natural yarns such as cotton. With respect to its thickness, the fabric could have a thickness of a single 1/50 (fifties) count yarn but could be, for example, about a 1/70 to 1/10 but preferably would be about a 1/60 to 1/30 count yarn (1/70 is thinner than 1/10), although other thicknesses may be suitably used. The fabric (in a non-folded combination, that is, one knitted layer) could be about 0.3 to 3 millimeters (mm) thick, preferably about 0.7 to 1.5 mm thick—although greater or lesser thicknesses could be used, provided the face seal allows for adequate fluid flow. The knitted face seal may be made from 1 to 10 yarn ends, preferably about 1 to 5 yarn ends. The number of yarn ends pertains to the number of yarn threads that are knitted together. The knitted fabric may be made such that there are about 1 gg to 20 gg, more preferably 12 gg to 18 gg. The notation “gg” pertains to the number of loops per inch. In a 16 gg machine, there are 16 needles per inch on the knitting machine. What is important is that the face seal is comfortable to wear and that it allows exhausted air to be rapidly purged from the interior gas space.

The knitted fabric may comprise approximately 15% of an elastane yarn such as Lycra™ and approximately 85% Notex™ yarn, an inherently flame-retardant yarn. The elastane adds elasticity to the inherently stretchy knitted Notex™, thereby avoiding the need for additional elastic materials on the face seal. Alternatively, the knitted material may comprise approximately to 20% elastic yarn, 45 to 55% Kanecaron™ yarn, and approximately 40 to 60% cotton yarn based on weight. Alternatively, flame-retardant and comfort fibers could be combined or a comfortable flame-retardant yarn could be used to provide flame-retardant yarn that is comfortable. For example, Kanecaron™ threads could be combined with cotton threads to form a flame-retardant/comfort yarn. A yarn that provides both flame retardance and comfort is referred to as a “combed flame-retardant/comfort yarn”. An elastic yarn could be knitted with such a combination yarn to provide a stretchably resilient fabric that is both comfortable and flame retardant. The elastomer may be, for example, a modified acrylic, a latex, or a combination thereof. Using such knitted materials, a comfortable breathable face seal can be achieved, which also allows air to properly exit the breathing zone or interior gas space of the helmet.

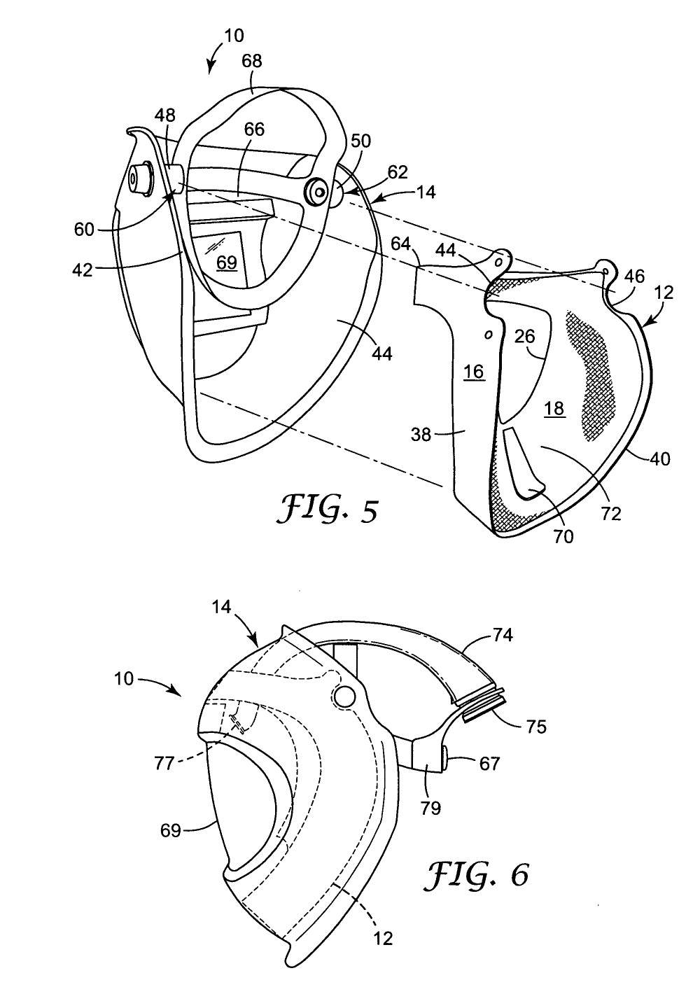

As also shown in FIG. 1, the knitted face seal 12 may have first and second zones 20 and 22, which zones define areas of first and second degrees of permeability. The first permeability zone 20 may have a greater resistance to airflow (or a higher pressure drop across it) so that air that exits the interior gas space is more readily channeled through the second permeability zone 22. There typically is more elastic yarn in the more permeable zone 22 than in the zone 20. This graduated or differentiated permeability feature is more thoroughly described in U.S. patent application Ser. No. ______, entitled Supplied Air Helmet Having Differentiated Permeability, filed on the same day as this application under Attorney Docket No. 60020US002. The benefit of differentiated permeability is that it can allow fluid to be selectively directed in flow across the interior side of the visor lens 69 (FIGS. 5 and 6) and that it may enable such directed flow in front of the face and under the chin of the wearer to help prevent contaminant backflow into the interior gas space.

FIG. 3 is a side view of the face seal 12, illustrating how the frame member 16 is constructed to define an angle α. The frame member 16 has a brow portion 21 and a chin portion 23, which portions 21 and 23 define an included angle α that is less than 90°, preferably about 50 to 80 degrees. Use of a frame member 16 that has such an angle α can allow a good fit to be achieved while prohibiting contaminant entry into the interior gas space.

FIG. 4 shows how the sealing member 18 can be secured to the frame member 16. This may be accomplished, for example, by sewing the knitted sealing member 18 to the frame member 16 at location 24. A strip of elastic material 25 may be used to help secure the fabric to the frame member 16. The elastic material 25 may be stitched or sewn to the frame member 16 through the sealing member 18. Because the fabric is generally porous, the elastic material 25 serves to preclude the stitching yarn from tearing the fabric when sewn to the frame member. Further, the knitted fabric 18 preferably has elastic qualities and is applied to the frame member in a stretched or extended condition. An elastic securement member 25 can be stretched contemporaneously with the sealing member material during the securement to the frame member 16. Securement of the fabric 18 to the frame member 16 in this manner enables the fabric to reside thereon in a taut condition. This pre-stretched or taut condition allows the fabric to snugly engage the wearer's face, particularly in the area near the peripheral inward edge 26. An elastic yarn may be included in the knit to render the fabric itself resiliently stretchable throughout a substantial portion thereof, particularly in the region where the face seal contacts the wearer's face. There is, thus, no need to include, for example, an elastic strip around the edge of the face seal, as has been done in the prior art. Use of an elastic yarn integral to the knot, eliminates a “ruffled effect” that may occur along the edge where the face seal contacts the wearer's face. Although the knitted fabric may be disposed on the frame member in a prestretched condition in accordance with this invention, it does not need to be 100% stretched. Typically, the sealing member 18 is installed at about 30 to 90% of its fully stretched condition, and thus is capable of being further stretched or expanded to snugly engage or accommodate various sized faces. In addition to sewing, the knitted fabric also may be secured to the frame member 16 using other mechanical or physical methods such as riveting, screwing, adhesive bonding, and the like.

FIG. 4 also shows how the knitted sealing member 18, is folded over to define an interior periphery 26 (see also FIG. 1). Use of a folded end or edge 26 along the interior periphery may further improve wearer comfort by eliminating or reducing a rough or more prominent edge that could otherwise exist. A knitted fabric that has a fold where the periphery of the seal member makes predominant contact with the wearer's face has been found to be particularly comfortable when in contact with a wearer's face. Opportunities for fraying also may be further reduced through use of the folded edge 26, which could also benefit wearer comfort. Thus, the fold 26 causes the sealing member 18 to not have a free or straight edge that could roughly contact a person's face. The fold 26 further creates first and second juxtaposed layers 28 and 30 for the sealing member 18. Layer 30 resides in direct contact with the wearer's face mask when the face shield 10 is worn. This double-layered construction can be beneficial in preventing contaminants from entering the interior gas space.

FIG. 5 illustrates how the face seal 12 can be secured to a visor 14. The frame member 16 of the face seal 12 has first and second side portions 38 and 40. The side portions 38 and 40 are pushed inward or towards each other so that they can be squeezed between respective first and second visor side portions 42 and 44. As indicated, the frame member 16 can be adapted to conform in response to manual pressure and to return towards an original configuration when that pressure ceases. The frame member 16 thus may be flexible or conformable to allow for its frictional placement within the more rigid visor 14. The frame member 16 has first and second receptacles 44 and 46, respectively, that are fashioned to reside against the spacer elements 48 and 50 at temporal locations 60 and 62. Once the frame member 16 is placed within the visor 14 such that receptacles 44 and 46 are juxtaposed against the hinge assembly at locations 60 and 62, the frame assembly 16 may be rotated counterclockwise until the front portion 64 of frame member 16 engages a third engagement point or shelf 66 on visor 14. When the front portion 64 of the frame member 16 is juxtaposed upon shelf 66, further rotational movement is prohibited, and the face seal 12 remains statically held within the face shield 14. Although the present drawings illustrate three frictional engagement points, the invention contemplates the use of further engagement points (e.g., 4, 5, 6 or more) if necessary or desirable. Alternatively, the frame member can be fixed to the visor by placing holes in the face seal frame that “click over” plastic rivets. These rivets may be permanently located on the visor or frame member to allow the frame to be clicked into place on the visor.

An example of a face seal that can frictionally engage a helmet visor is described in detail in U.S. patent application Ser. No. ______, entitled Frictionally Engaged Supplied Air Helmet Face Seal, filed on the same day as this application under attorney docket number 60021US002. This face seal is particularly beneficial in that it can be attached to the visor through use of frictional engagement without using additional fastening equipment. Another example of a face seal that could possibly be used is shown in U.S. Pat. No. 6,016,805 to Burns et al.

To don the helmet 10, the wearer places the crown member 68 on their cranium and rotates the visor 14 downwardly such that it resides directly in front of the wearer's face. The wearer can then look through window 69. If the helmet is used for welding purposes, the window can be an auto-darkening lens (ADL) that darkens immediately in response to light from a welder's torch, (see, for example, U.S. Pat. Nos. 6,097,451 and 5,825,441, issued to Hornell and Palmer). The wearer then pulls on tab 70 to draw the chin portion 72 of sealing member 18 underneath their chin. The remainder of the periphery 26 of the sealing member 18 draws tightly against the wearer's forehead and cheek regions. A breathing zone or interior gas space is thus created, defined by the wearer's face, the sealing member 18, and the face shield or visor 14. As indicated, the knitted fabric can be integrally porous to allow air to be purged from the interior gas space. Despite the generally porous nature of a knitted fabric, contaminants are precluded from entering the interior gas space because of the positive pressure that generally exists within it during use. In operation, clean air is supplied to the interior gas space under pressure from a powered air supply source. Examples of these types of devices are shown in U.S. Pat. Nos. 6,279,572B1, 6,250,299B1, 6,014,971, 5,125,402, 4,965,887, 4,462,399, and 4,280,491. Examples of blowers that may be used in connection with a supplied air system for directing air into the interior gas space are shown in U.S. Pat. Nos. 6,575,165B1 and D449,099S. A flow sensor may be used on the supplied air helmet to provide an indication of when air flow into the breathing zone falls below a safe level—see U.S. Pat. No. 6,615,828 B1 to Petherbridge. In addition, a non-volatile memory device may be attached to the filter element to keep a record of the filter element's usage—see U.S. Pat. No. 6,186,140 B1 to Hogue.

As shown in FIG. 6, the air may be channeled into the interior gas space via an air duct 74 that is in fluid communication with the powered air supply source. The duct 74 has an inlet port 75 and an outlet port 77. An air duct that could be used to direct air into the interior gas space of the helmet is further shown in U.S. patent application Ser. No. 29/202,969, entitled Air Duct, filed on Apr. 7, 2004 and now U.S. patent Ser. No. ______ to Hind et al. The air duct 74 has an air inlet 75 and an air outlet 77 and is supported by the crown member 68. The inlet 75 is connected to the clean air source, and the outlet 77 is disposed between the sealing member 18 and the wearer's forehead (not shown). As indicated, air flow exits the interior gas space through, for example, pores that may be present in the sealing member 18 (FIGS. 1 and 3). The air duct 74 can be fixed to the crown member 68 in two places. The rear of the air duct 74 can be held a fixed distance from the back of the head harness 68 by a stamped out plastic part 79. An adjustment knob 67 can be provided to alter the circumference of the crown member 68 so that it fits various sized heads. The front of the air duct 74 can be stapled to the front of the head harness 68 at the brow. In this way, the air duct 74 is prevented from moving or wobbling when in use. At the brow, the air duct 74 passes between the face seal 12 and the head harness 68. An elastomeric face seal material can allow the face seal to form a good seal around the air duct. The inlet would be connected to the clean air source, and the outlet can be disposed between the face seal and the visor. When using a powered air supply source, the air is powered or forced through an air filter before being directed into the interior gas space. The air filter may be contained in a housing or cartridge that is supported on a belt that is worn about the wearer's waist—see U.S. Pat. No. 6,575165 to Cook et al.

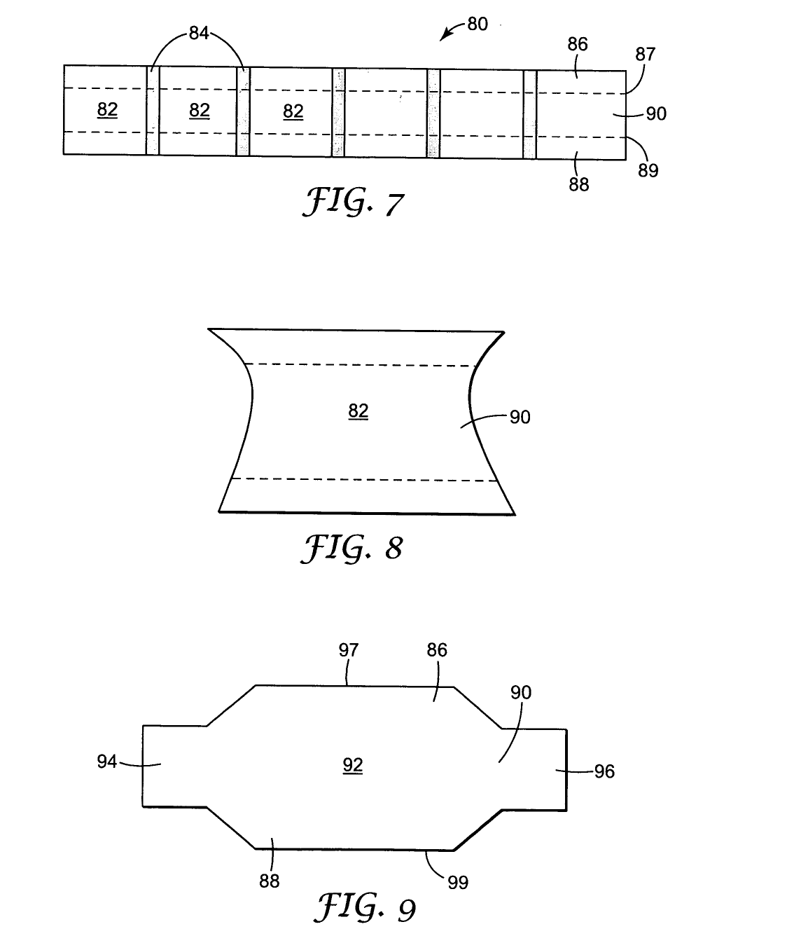

FIG. 7 shows an elongated strip of knitted material 80, which strip 80 includes a series of blank segments 82 for forming multiple knitted sealing members. Each segment 82 is separated by a decomposable portion 84. Both segments 82 and 84 are knitted together such that the strip of material forms a continuously-knitted elongated sheet 80. The sheet 80 may have a uniform knit throughout, but it preferably includes zones that have different degrees of elasticity. Zones 86 and 88 are located above and below the dashed lines 87 and 89, respectively, and preferably have little or no elastic material or yarn in the knit. Zone 90, has relatively more elastic material in it than zones 86 and 88. Using a knitted material that has variable zones of elasticity can be beneficial to the final product's construction as discussed above with the reference to FIGS. 1 and 2. Preferably zones 86 and 88 contain about to 5% elastic yarn, more preferably about to 1%, and zone 90 contains about 2 to 10% elastic yarn, more preferably about 3 to 7%, based on the fabric weight.

In preparing a knitted face seal in accordance with the present invention, the elongated strip of knitted material 80 may be exposed to steam, which steam causes spaced zones 84 to decompose. These decomposable portions 84 may be made predominantly from a water-soluble or “seaweed” derived or man-made yarn, such as Grilon™ available from EMS-Chimie AG of Switzerland. Upon exposure to a sufficient amount of steam, the water-soluble yarn decomposes, and the elongated sheet 80 forms a series of blanks 82.

FIG. 8 illustrates one of the formed blanks 82. As shown, the segment 82 is “waisted”. This waisted effect occurs because there is a larger amount of elastic in the central zone 90. When the sheet material 80 of FIG. 7 is exposed to steam to separate the segments 82, the elastic material may become cured, causing it to “bunch up” and causing segments 82 to become waisted.

The waisted segments 82 from FIG. 8 are each cut into the shape shown in FIG. 9. Each cut segment has opposing tabs 94 and 96. These tabs are sewn together to make a tube. The opposing ends 97 and 99 of the tube are then joined together and are sewn to the frame member 16 of the face seal 10 as discussed above with reference to FIG. 4. The elastic zone 90 thus resides towards the peripheral end 26 of the sealing member 18 (FIGS. 1 and 3). The less stretchable or nonelastic zone 20, as defined by zones of material 86 and 88, thus becomes located closer to the fixed portion of the face seal as noted by numeral 24 in FIG. 3. Not only may portions 86 and 88 contain less elastic material than portion 90, but portions 86 and 88 may also include a tighter or more dense knit so that zone 20 (as shown in FIGS. 1 and 3) has substantially less air permeability than zone 22. Zone 22 thus would be more porous or permeable and would have a lower pressure drop than zone 20. Zone 22, accordingly, would form a path of least resistance for air that is to be exhausted from the interior gas space of the supplied air helmet. The pressure drop across the face seal as a whole typically is about 10 to 200 Pascals, and more typically about 20 to 110 Pascals. The high permeability zone preferably has a pressure drop of about 10 to 100 Pascals, more preferably about 20 to 70 Pascals. Low permeability zone 20 preferably has a pressure drop of about 90 to 200 Pascals across it, more preferably about 120 to 180 Pascals. The pressure drop can be measured by clamping the face seal over a 60 millimeter diameter hole using a pair of pneumatic chucks. A manometer measures the pressure on each side of the face seal using a flow rate of 85 liters per minute.

The air flow rate across the materials of the face seal typically is about 5 to 200 cm³/s/cm², and more typically about 20 to 150 cm³/s/cm². The high permeability zone 22 preferably has an air flow rate of 85 to 200 cm³/s/cm², more preferably about 100 to 150 cm³/s/cm². Low permeability zone 20 preferably has an air flow rate of about 5 to 80 cm³/s/cm² across it, more preferably about 20 to 70 cm³/s/cm². Air flow rates can be measured using the test method described in ASTM D737-96, Standard Test Method for Air Permeability of Textile Fabrics.

A controlled flow of air from the exit port 77 (FIG. 5) across the face of the wearer may be achieved. The clean air that enters the breathing zone from port 77 (FIG. 6) exits the interior gas space predominantly through the more porous zone 22 (FIGS. 1 and 4). Because of the increased pressure within the interior gas space, it is more difficult for contaminants to enter the breathing zone of the supplied air helmet. The provision of a sealing member that has a differentiated permeability or differential air flow allows for a managed movement of air within the breathing zone. Pressurized air can sweep across the face or breathing zone of the wearer while precluding the inadvertent influx of contaminants into the interior gas space.

In an alternative method for making the knitted face seal, the yarn may be knitted into a long hollow cylinder, which then can be cut into a series of shorter loops. Each of these shorter loops then can be doubled over and sewn to a frame member to form a face seal. Using this method, it may be possible to make a face seal without any exposed seams. A seamless construction may further improve wearer comfort. The face seal porosity may be controlled by altering the density of the knits, by adding additional material to the face seal loop, and by controlling the tension of the material that is sewn to the plastic frame.

In addition to the powered air systems mentioned above, the present invention also could be used in conjunction with a compressed air system such as a self-contained breathing apparatus (SCBA) that has a tank of air or oxygen, typically under pressure, for supplying clean air to a person. Examples of SCBA systems are shown in the following U.S. Pat. Nos. 6,478,025, 4,886,056, 4,586,500, and 4,437,460. For purposes of construing this invention, a compressed air system is considered to be a supplied air system. Further, the inventive supplied air systems may be used not only in conjunction with welding helmets or welding environments but may also be used, for example, in helmets fashioned for surgical environments and clean air rooms—see, for example, U.S. Pat. Nos. 4,901,716, 4,055,173, 4,019,508, and 3,955,570.

The following Example has been selected merely to further illustrate features, advantages, and other details of the invention. It is to be expressly understood, however, that while the Example serves this purpose, the particular ingredients and amounts used, as well as other conditions and details, are not to be construed in a manner that would unduly limit the scope of this invention.

### EXAMPLE

The face seal sealing member was made from a rectilinear 1&1 rib knitted blank that had been cut and sewn to achieve the desired shape and configuration. The blank was made from three yarns: a combined flame-retardant/comfort yarn, an elastic yarn, and a water soluble yarn. The combined flame-retardant/comfort yarn contained Kanecaron™ fiber, Protex-M, and cotton fiber. Relative to each other, the Kanecaron™ fiber was used at 55 weight %, and the cotton fiber was used at 45 weight %. The elastomeric yarn was 200 decitex and contained an elastane, LycraTm, and crimped nylon at 62 wt. % and 38 wt. %, respectively. These two yarns functioned as the primary structural element in the knitted blank. The combined flame-retardant/cotton yarn and the elastic yarn were both dyed blue. The flame-retardant/comfort yarn was waxed as well. Content of elastic yarn in the blank varied along the length of the blank with the highest amount of yarn in the centre of the blank, reducing to no elastic yarn at the top and bottom of the blank. To enable a series of blanks to be produced in a continuous manner, a row of water soluble yarn (Grilon™, EMS-Chimie AG, Switzerland) was knitted at the end of each blank. The continuous fabric was knitted on a 16 gg (16 needles per inch or 6.3 needles/cm) power flat machine that had 620 needles in use. When subjected to steam treatment, the water-soluble yarn dissolved to form the individual blank from the continuous-length knit. The blank included five graduated areas:

Area 1 was a stiffer knit that had an extra yarn end and was designed to allow clean air to be exhausted from the headtop. It contained three ends of the Kanecaron™/cotton yarn, had 24 cycles and 48 rows. Area 2 was a transition area between the exhaust area 1 and the face seal area 3. It comprised two ends of 1/50 count yarn and 48 rows that contained tucked Lycra™ yarn, used one in every 4 rows. Area 3 was the face contact area and was designed to be comfortable against the skin. Area 3 comprises two ends of 1/50 count yarn and had 11 cycles and 88 rows, in which one in every two rows was tucked with Lycra™ yarn. Area 4 was a transition area between the face contact area and the exhaust area and had the same structure as area 2. Area 5 also was an exhaust area and had the same structure as area 1.

With a greater percentage of elastic yarn near the center of the blank, the side edges of the blank tapered or ‘waisted’ as the edge was followed to the center of the blank. To form the face seal of the invention, the blank edges were sewn together using a type 301 lockstitch to form a cylinder. The cylinder was then folded such that the edges of the two open ends could be sewn together, adding a type 514 four thread overlock. These edges were also sewn to a supporting plastic frame, which was a presscut 1 mm thick black polypropylene plastic. The knitted material was secured using a 301 lockstitch sewing machine that had 3-4 stitches per centimeter. Finally a leather tab was sewn to the knitted material.

This invention may take on various modifications and alterations without departing from the spirit and scope thereof. Accordingly, it is to be understood that this invention is not to be limited to the above-described, but it is to be controlled by the limitations set forth in the following claims and any equivalents thereof.

It is also to be understood that this invention may be suitably practiced in the absence of any element not specifically disclosed herein.

All patents and patent applications cited above, including those in the Background section, are incorporated by reference into this document in total.

What is claimed is:

1. A supplied air helmet that comprises:
(a) a visor; and
(b) a face seal that is secured to the visor and that includes a sealing member that comprises a knitted fabric, the knitted fabric being porous in at least some regions and being disposed in a location where the sealing member makes contact with the wearer's face.

2. The supplied air helmet of claim 1, wherein the knitted fabric has a fold line that defines an inner periphery of the sealing member.

3. The supplied air helmet of claim 1, wherein the knitted fabric contains an elastic yarn.

4. The supplied air helmet of claim 3, wherein the elastic yarn is present in the knit at about 1 to 10 weight % by weight of the fabric.

5. The supplied air helmet of claim 3, wherein the knitted fabric contains a flame-retardant yarn and a comfort yarn or a combined flame-retardant/comfort yarn.

6. The supplied air helmet of claim 5, wherein the flame-retardant yarn or combined flame-retardant/comfort yarn is used in the fabric at about 80 to 99%.

7. The supplied air helmet of claim 1, wherein face seal comprises a frame member from which the sealing member extends radially inward therefrom.

8. The supplied air helmet of claim 7, wherein the frame member is adapted to be conformed in response to manual pressure and to return to an original configuration when that pressure ceases.

9. The supplied air helmet of claim 7, wherein the frame member is non-integral with a visor and is capable of being manually removed therefrom.

10. The supplied air helmet of claim 7, wherein the sealing member includes a fold line that defines an inner periphery of the sealing member.

11. The supplied air helmet of claim 10, wherein the knitted fabric includes elastomeric yarn in the vicinity of the fold line.

12. The supplied air helmet of claim 1, wherein the face seal includes a frame member that is made from a fluid impermeable thin solid plastic.

13. The supplied air helmet of claim 12, wherein the plastic frame is about 0.5 to 1.5 millimeters thick and is made from plastic.

14. The supplied air helmet of claim 1, wherein the face seal includes a frame member that is integral to the visor.

15. The supplied air helmet of claim 1, wherein essentially the whole knitted fabric is inherently porous.

16. The supplied air helmet of claim 1, wherein the knitted fabric comprises elastic yarn, flame-retardant yarn, and comfort yarn or elastic yarn and a combined flame-retardant/comfort yarn.

17. The supplied air helmet of claim 16, wherein the elastic yarn is more prevalent adjacent an inner peripheral edge of the sealing member.

18. The supplied air helmet of claim 16, wherein the flame-retardant yarn is inherently flame retardant.

19. The supplied air helmet of claim 1, wherein the knitted fabric has 12 to 18 gg.

20. The supplied air helmet of claim 12, wherein the frame member has a brow portion and a chin portion that are offset from each other at an angle a of about 50 to 800.

21. The supplied air helmet of claim 12, wherein the knitted fabric has elastic qualities and is secured to the frame member in a stretched condition.

22. The supplied air helmet of claim 21, wherein the sealing member is attached to the face seal at about 30 to 90% of its fully stretched condition at the location of attachment.

23. The supplied air helmet of claim 1, wherein the knitted face seal exhibits a pressure drop of about 20 to 110 Pascals.

24. The supplied air helmet of claim 1 being a welding helmet.
